(19) United States
(12) Patent Application Publication
(10) Pub. No.: US 2006/0190017 A1
(43) Pub. Date: Aug. 24, 2006

(54) Fibrin sealants and platelet concentrates applied to effect hemostasis at the interface of an implantable medical device with body tissue

(76) Inventors: John St. Cyr, Loon Rapids, MN (US); Nancy Rakow, Bethel, MN (US); Linda M. Shecterle, Plymouth, MN (US)

Correspondence Address: Katharine A. Jackson Huebsch;Medtronic, Inc., 7601 Northland Drive, Minneapolis, MN, 55428, US

(21) Appl. No.: 11/282,276

(22) Filed: Nov. 18, 2005

Related U.S. Application Data
(60) Provisional application No. 60/629,434, filed on Nov. 19, 2004.

(51) Int. Cl. A61F 2/04

(52) U.S. Cl. 606/151; 623/1.36

(57) ABSTRACT

Surgical methods of and kits for applying and stabilizing a mass of fibrin sealant or platelet concentrate at the site of surgical attachment of an implantable medical device to effect hemostasis to stem internal bleeding at the site of surgical attachment are disclosed. A mass of fibrin sealant or platelet concentrate is applied onto a porous fabric, whereby the mass is supported in the interstices or pores of the fabric, and the supported mass is applied against the site of high pressure blood leakage. The supported mass achieves hemostasis as it does not wash away from the site. The present invention is particularly useful to effect hemostasis at sutures and suture holes extending through thin-walled tissue valves and grafts when such tissue valves or grafts are sutured in place, particularly at high blood pressure sites as at the valve annulus of the aortic valve or the aorta.

### PRIORITY CLAIM

This application claims priority under 35 U.S.C. § 119(e) to U.S. Provisional Application Ser. No. 60/629,434, filed Nov. 19, 2004, the entire contents of which are incorporated herein by reference.

### FIELD OF THE INVENTION

This invention relates to surgical methods of stemming internal bleeding at the site of surgical attachment of an implantable medical device, and more particularly to improved methods of applying and stabilizing a mass of fibrin sealant or platelet concentrate at the site of surgical attachment of an implantable medical device to effect hemostasis.

### BACKGROUND

A wide range of implantable medical devices are implanted in the body and in certain cases are sutured to body tissue to fix the implantable medical device in place. A number of implantable medical devices are sutured in place to or within blood vessels to repair the vessel or to replace a valve. Surgeons strive to effect implantation of implantable medical devices with minimal loss of blood.

In particular, implantable heart valve prostheses or prosthetic heart valves have been used to replace various diseased or damaged native aortic valves, mitral valves, pulmonic valves and tricuspid valves of the heart. Heart valves are most frequently replaced due to heart disease, congenital defects or infection. The aortic valve controls the blood flow from the left ventricle into the aorta, and the mitral valve controls the flow of blood between the left atrium and the left ventricle. The pulmonary valve controls the blood flow from the right ventricle into the pulmonary artery, and the tricuspid valve controls the flow of blood between the right atrium and the right ventricle. Prosthetic heart valves can be used to replace any of these naturally occurring valves, although repair or replacement of the aortic or mitral valves is most common because they reside in the left heart chambers where the pressure loads are higher and valve failure is more common. Generally, prosthetic heart valves are either bioprostheses or mechanical heart valve prostheses.

Modern mechanical heart valve prostheses (hereafter “mechanical valves”) are typically formed of an annular valve seat in a relatively rigid valve body and an occluding disk or pair of leaflets that are movable through a prescribed range of motion between a closed, seated position against the annular valve seat blocking blood flow and an open position allowing blood flow. Such mechanical valves are formed of blood compatible, non-thrombogenic materials, typically comprising pyrolytic carbon and titanium. Hinge mechanisms and struts entrap and prescribe the range of motion of the disk or leaflets between the open and closed positions.

The bioprostheses (hereafter “tissue valves”) fall into two groups, homografts recovered from human cadavers and xenografts harvested from animal hearts. The most widely used tissue valves include some form of synthetic support, referred to as a “stent,” although so-called “stentless” tissue valves are also available. The most common tissue valves are constructed using an intact, multi-leaflet, harvested donor tissue valve, or using separate leaflets cut from bovine (cow) pericardium, for example. The most common intact donor tissue valve used for stented and stentless valves is the porcine (pig) aortic valve. Porcine tissue valves include the entire porcine valve in an intact configuration harvested from a single pig or in some cases, cusps or leaflets from up to three different heart valves excised from pigs then sewn back together. Exemplary tissue valves formed of swine valve leaflets mounted to struts of a stent are those disclosed in U.S. Pat. Nos. 4,680,031, 4,892,541, and 5,032,128 as well as the MEDTRONIC® Hancock II® and Mosaic® stented tissue valves. Stentless tissue valves, e.g., the MEDTRONIC® Freestyle® stentless aortic root bioprostheses and the stentless tissue valve disclosed in U.S. Pat. No. 6,797,000 are formed from treated integral swine valve leaflets and ascending aorta structure.

Mechanical valves and tissue valves are intended to be sutured to the “native annulus” or a peri-annular area of a natural heart valve orifice after surgical removal of damaged or diseased natural valve structure from the patient's heart (referred to hereafter for convenience as the valve annulus). The suture stitches extend through the valve annulus and a fabric sewing ring of mechanical heart valves and stented tissue valves thereby drawing the sewing ring against the tissue of the valve annulus at a site of surgical attachment or interface. Sewing rings typically comprise a fabric strip made of synthetic fiber that is biologically inert and does not deteriorate over time in the body, such as polytetrafluoroethylene (e.g., “Teflon” PTFE) or polyester (e.g., “Dacron” polyester), that is knitted or woven having interstices permeable to tissue ingrowth. The valve body or stent typically has a circular or ring-shaped sidewall shaped to mate with an inner sidewall of the sewing ring, and the sewing ring has an annular outer surface. In some cases, the sewing ring fabric is shaped to extend outward to provide a flattened collar or skirt that can be applied against and sutured to the native tissue annulus, as shown for example in U.S. Pat. Nos. 3,997,923 and 4,680,031.

Bleeding of high pressure blood can occur post-operatively along the sutures or through the suture holes at the sutured site or interface when such a prosthetic valve is sutured in place of a diseased or defective aortic heart valve. The thickness and porosity of the sewing ring fabric can influence how long bleeding occurs with consequent loss of blood. With relatively thick or dense suture rings, the loss of blood is relatively minor and halts after a short time period as coagulation in the suture holes and along the sutures takes place. Nevertheless, it may be necessary to provide a chest drainage catheter exiting through the skin to drain blood pooling about the surgical site for a period of days and to monitor the loss of blood.

Suture rings are not incorporated onto the above-referenced MEDTRONIC® Freestyle® stentless aortic root bioprostheses and the stentless tissue valve disclosed in U.S. Pat. No. 6,797,000, for example. Instead, protective reinforcement fabric bands of porous polyester fabric are sutured about the inflow end of the tubular valve body, and sutures are passed through the fabric and valve annulus to effect fixation. A number of surgical preparations of the outflow end attachment to the aorta are possible. The surgeon may trim the outflow end of the valve body to attach it to the prepared aorta in a variety of ways. The multiple suture stitches create a relatively large number of suture perforations of the valve body and reinforcement fabric resulting at times in excessive bleeding and post-operative blood loss, which can complicate a patient's recovery. Patients that suffer significant blood loss may in particular require a transfusion or re-operation because of excessive blood loss.

A similar problem may arise when it is necessary to replace a length of the ascending aorta with a flexible fabric graft, e.g., the Gelweave™ Valsalva™ aortic graft sold by Vascutek Ltd, Inchinnan, UK or the Hemashield™ aortic graft sold by Boston Scientific Corporation, Natick, Mass. The degeneration of natural heart valves through a disease process is sometimes accompanied by degeneration of blood vessels extending from the heart valve, particularly an aneurysm of the ascending aorta coupled to the aortic valve. Consequently, both the aortic valve and a segment of the ascending aorta must be replaced at the same time. Certain grafts have fabric pores sealed with collagen or gelatin to inhibit significant blood leakage through the pores at the time of surgery. After blood flow is re-established, the sealing material is absorbed and replaced with a fibrin layer that grows into the graft material. However, blood leakage through the suture holes made through the graft fabric typically occurs until the blood seals the holes.

The problem of internal bleeding has caused complications in surgery or after traumatic damage for generations, as described in U.S. Pat. No. 4,128,612, for example. Different techniques have been used to control bleeding, i.e., to achieve hemostasis, including sutures, ligatures, clamps or staples applied to hold severed tissue layers together or to close severed blood vessels, application of cyanoacrylate-based tissue adhesives, and the application of electro-cautery, electro-surgery or argon beam coagulation to the site of bleeding. In addition, various forms of dressings, gauzes, felts, knitted fabrics and collagenous sponges and pads have been used to aid in clotting or otherwise control the flow of blood. Various forms of absorbable hemostatic agents, e.g., foamed gelatin, knitted oxidized regenerated cellulose, and other coagulant entities including “gel foam” gelatin foam and Surgicel® oxidized regenerated cellulose hemostatic agents are available in dry sheet form to be cut or broken into smaller sizes and topically applied at a bleeding site. As stated in the '612 patent, such oxidized regenerated cellulose and gelatin foam hemostatic agents are wetted with saline at the time of use and are difficult to apply as the wetted hemostatic agent is limp and somewhat pasty or gelatinous so that it may stick to instruments and gloved fingers rather than remain at a bleeding site. The applied oxidized regenerated cellulose and gelatin foam hemostatic agents may be washed away from the site by significant bleeding.

The '612 patent further discloses a tissue absorbable synthetic polymeric fiber hemostatic felt that is heat compacted on at least one surface. The compaction and heat embossing aid in causing the hemostatic surgical felt to adhere to the surface of a wound, and because it adheres so closely due to capillary hemorrhage is usually effectively controlled. If a major blood vessel is severed, the hemostatic felt may be floated from the surface of a wound, but for many procedures, such as the excision of a part of a liver or neurosurgery, the adherence is such as to promptly cause hemostasis. The compacted hemostatic felt is preferably thick enough and compacted enough that blood does not flow from the outer surface. Because of the non-absorbable characteristic of the hemostatic felt, it is left in place when a wound is closed to provide effective blood flow control during the surgical procedure and to minimize subsequent bleeding. No attempt is made to remove hemostatic felt, since the removal could cause renewed bleeding.

Surgical hemostasis is also achieved using blood or plasma based tissue adhesives or fibrin sealants. The composition and uses of various forms of fibrin sealants are described in an article entitled “Blood Bank and Commercial Fibrin Sealant” in the Winter 1998 issue of the newsletter of the Tissue Adhesive Center of the University of Virginia. Such blood bank and commercial fibrin sealants are made from pooled human blood or topical cryoprecipitate and other materials including bovine and pooled human thrombin. As stated therein, such fibrin sealant has been used in a variety of applications, including achieving hemostasis along suture lines or at the site of vascular anastomoses, sealing vascular conduits and grafts to avoid leakage from interstices of prosthetic materials, and controlling diffuse mediastinal bleeding with notable reductions in postoperative chest tube bleeding and transfusion requirements.

It is also known to topically apply platelet concentrates, e.g., autologous platelet gel derived from the patient's own blood, at an incision or injury to encourage coagulation of the patient's blood and thereby halt bleeding. Use of the patient's own blood is highly attractive in that it avoids any adverse foreign body reactions and other potential complications, including viral transmission of hepatitis and HIV that might accompany use of blood bank and commercial fibrin sealants. Commonly assigned U.S. Pat. No. 6,596,180 describes a centrifuge system for the formation of an autologous platelet sealant or gel wherein all of the blood components for the gel are derived from a patient to whom the gel is to be applied. First a platelet rich plasma and a platelet poor plasma are formed by centrifuging a quantity of anticoagulated whole blood that was previously drawn from the patient. The platelet rich plasma or platelet poor plasma is then automatically drawn out of the centrifuge bag and proportioned into separate chambers in a dispenser. The first portion is activated where a clot is formed and thrombin is obtained. The thrombin is then later mixed with the second portion to obtain a platelet gel. This process can be practiced employing the Magellan™ Autologous Platelet Separator System sold by the assignee of the present invention.

A small amount of whole blood (approximately 50 to 120 milliliters) is drawn, either pre-operatively or in the operating room, into a syringe containing a citrate-phosphate-dextrose adinine. The blood is then centrifuged by using a variable-speed centrifuge autotransfusion machine or portable machine, to separate the buffy coat suspended in plasma above the red blood cell layer and below the platelet-poor plasma fraction. This is the platelet concentrate used for Platelet Gel. Other important factors in quality of Platelet Gel are platelet viability and percent retained in the procedure. While white cell content increases 125% with selection for lymphocytes and monocytes, the inclusion of platelets and white cells appears have several beneficial aspects. White cells confer additional healing cytokines while providing antibacterial activity. On activation with thrombin/calcium to form a coagulum, the platelets interdigitate with the forming fibrin web, developing a gel with adhesiveness and strength materially greater than the plasma alone. Thrombin/calcium also causes platelets to immediately release highly active vasoconstrictors, including beta thromboxane, serotonin and PDGF.

It has been found that the high pressure of blood within chambers or conduits can cause the blood to leak through suture holes, e.g., through suture holes through stentless tissue valve walls or aortic grafts, simply washes away the topically applied fibrin sealant or platelet gel before it can act to coagulate the escaping blood. The minute confines and spaces about the sutures may also complicate application of the fibrin sealant or platelet gel to the site.

It would be desirable to provide an inexpensive, relatively simple and easy to practice method of stabilizing the applied fibrin sealant or autologous platelet gel in the flow of relatively high pressure blood to aid in rapid coagulation and to diminish blood loss. Similarly, it would be desirable to employ the same method in other areas of the body to stabilize fibrin sealant or autologous platelet gel applied against an organ or vessel or tissue surface to aid rapid coagulation and diminish blood loss due to trauma or surgical intervention.

### BRIEF SUMMARY OF THE INVENTION

Therefore, the present invention provides a method of supporting, scaffolding, latticing or otherwise restraining a mass of platelet concentrate or fibrin sealant at a site of blood leakage to achieve hemostasis. Preferably, the method of the present invention is practiced employing autologous platelet gel to achieve hemostasis.

In preferred embodiments, the method of the present invention is achieved by obtaining a mass of fibrin sealant or platelet concentrate, applying the mass topically onto a porous fabric, whereby the topically applied mass is supported in the interstices or pores of the fabric, and applying the supported mass against the site of high pressure blood leakage. The supported mass achieves hemostasis as it does not wash away from the site.

The fabric may comprise a porous material that may be flexible or packable at the site of bleeding, including one of foamed gelatin, knitted oxidized regenerated cellulose, and other coagulant entities. In preferred embodiments, the porous fabric comprises a surgical felt formed of bio-compatible materials that may or may not be absorbable over time.

The present invention is advantageously employed to effect hemostasis at sutures and suture holes extending through thin-walled tissue valves and grafts when such tissue valves or grafts are sutured in place, particularly at high blood pressure sites as at the valve annulus of the aortic valve or the aorta. The present invention may also be advantageously employed at surgical sites or traumatic injury sites to stem bleeding.

Advantageously, the method of the present invention may be practiced by providing a kit of pre-formed porous fabric strips to be supplied with implantable medical devices, e.g., the aforementioned tissue valves and grafts.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic illustration of the human heart in partial cross section depicting the aortic valve and ascending aorta;

FIG. 2 is schematic illustration of a stentless tissue valve that is adapted to be sutured to a valve annulus of the heart of FIG. 1, particularly to replace a dysfunctional aortic valve;

FIG. 3 is a schematic illustration of the replacement of an aortic valve with a stentless tissue valve employing the full-root surgical technique;

FIG. 4 is a schematic illustration of the replacement of an aortic valve with a stentless tissue valve employing the root-inclusion surgical technique;

FIG. 5 is a schematic illustration of the replacement of an aortic valve with a stentless tissue valve employing the complete subcoronary surgical technique;

FIG. 6 is a schematic illustration of the replacement of an aortic valve with a stentless tissue valve employing the modified subcoronary surgical technique;

FIG. 7 is a schematic illustration of the replacement of a section of the ascending aorta with a graft;

FIG. 8 is a schematic illustration of a strip of surgical felt supporting a mass of fibrin sealant or platelet concentrate; and

FIG. 9 is a flowchart illustrating the method of providing hemostasis at a surgical site, e.g., the surgical sites of FIGS. 2-8, with a strip of surgical felt supporting a mass of fibrin sealant or platelet concentrate.

### DETAILED DESCRIPTION

In the following detailed description, references are made to illustrative embodiments of methods and apparatus for carrying out the invention. It is understood that other embodiments can be utilized without departing from the scope of the invention. As noted above, the present invention may be advantageously employed at surgical sites or traumatic injury sites to stem bleeding.

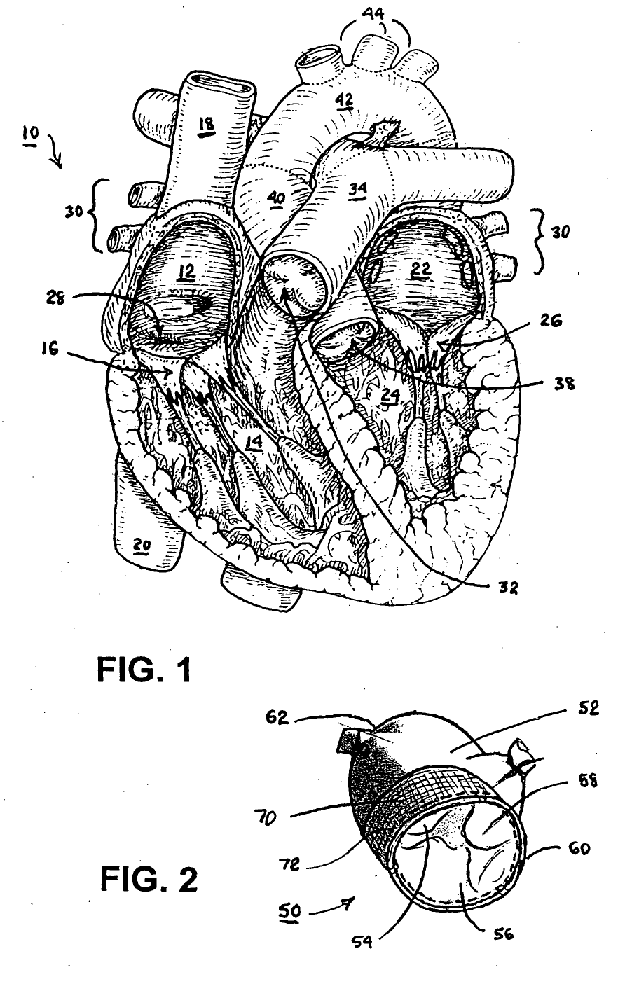

The heart 10 depicted in FIG. 1 comprises two right heart (pulmonary heart) chambers and two left heart (systemic heart) chambers. The pulmonary heart includes the right atrium 12, the right ventricle 14, and the tricuspid valve 16 separating the right atrium 12 and right ventricle 14. The systemic heart includes the left atrium 22, the left ventricle 24 and the bicuspid or mitral valve 26 separating the left atrium 22 and the left ventricle 24. Cardiac cycles are marked by synchronous contraction (systole) and relaxation (diastole) of the atria and ventricles. At the beginning of a cardiac cycle, the atria 12 and 22 briefly contract, the ventricles 14 and 24 contract shortly thereafter, and then the atria and ventricles relax between contractions.

When relaxed, the tricuspid valve 16 is closed and the right atrial chamber of the thin-walled right atrium 12 fills with deoxygenated venous blood draining from the body through the superior vena cava 18 and the inferior vena cava 20, and from the coronary sinus 28, which drains the coronary vessels. Similarly, when relaxed, the bicuspid valve 26 is closed, and the left atrial chamber of the thin-walled left atrium 22 fills with oxygenated or arterial blood draining from the lungs through the pulmonary veins, collectively designated 30.

When the right and left atria 12 and 22 contract, deoxygenated blood in the right atrial chamber and oxygenated blood in the left heart chamber is pumped substantially simultaneously through the tricuspid and bicuspid valves 16 and 26 at a pressure of about 5 mm Hg into the right and left ventricles 14 and 24. When the right ventricle 14 contracts, the flaps of the tricuspid valve 16 are closed and the flaps of the pulmonary semiluminar valve 32 are opened. The right ventricular blood is pumped through the pulmonary valve 32 and the pulmonary trunk and arteries, collectively designated 34, to the right and left lungs. Similarly, when the left ventricle 24 contracts, the flaps of the bicuspid valve 26 are closed and the flaps of the aortic semiluminar valve 38 are opened.

The left ventricular blood is pumped through the aortic valve 38, the ascending aorta 40 into the aortic arch 42 and distributed through the coronary arteries, collectively designated 44, and to arterial system 46 of the body. When the right and left ventricles relax, the flaps of the pulmonary and aortic valves 32 and 38 close preventing reflux of deoxygenated and oxygenated blood into the respective right and left ventricles 14 and 24.

During systole, the right ventricle 14 the pumps deoxygenated blood to the lungs via the pulmonary trunk 34 at a pressure of about 25 mm Hg, and the left ventricle 24 pumps oxygenated blood into the ascending aorta 40 at a pressure of about 120 mm Hg. The thicker, more muscular, walls of the left ventricle 24, compared to the walls of the right ventricle 14, accomplish this pressure difference. Consequently, oxygenated blood pumped through the aortic valve 38 into the ascending aorta 40 into the aortic arch 42 is at relatively high pressure during systole.

Cardiac diseases that affect the aortic valve 38 and/or the ascending aorta 40 and the aortic arch 42 and arteries branching therefrom significantly compromise cardiac function and lead to disability or death. Repair or replacement of a dysfunctional aortic valve 38 with a prosthetic valve of the types described above is a relatively common, albeit critical, surgical procedure. Similarly, the replacement of all or a section of the ascending aorta and the aortic arch that exhibit an aneurysm is a necessary and critical surgical procedure.

As described above, a dysfunctional aortic valve 38 is frequently replaced with a stentless tissue valve that involves removal of the diseased valve structure and preparation of remaining valve annulus and ascending aorta to receive the inflow and outflow ends of the tissue valve. An exemplary stentless tissue valve 50 depicted in FIG. 2 comprises an entire, full root length, harvested and cured porcine valve or xenograft in an intact configuration. Alternatively, the stentless tissue valve 50 may be formed from full root length sections, with intact cusps, of up to three different heart valves excised from pigs that are sewn back together as shown in the above-referenced '007 patent.

The stentless tissue valve comprises a valve wall 52 having three cusps 54, 56, 58 disposed within the valve lumen, the valve wall extending between an inflow end 60 and an outflow end 62. A porous fabric band 70 is sewn about the inflow end to strengthen and isolate the cured porcine tissue from myocardium when the inflow end 60 is sutured to the valve annulus. Surgeon's flags are marked about the porous fabric band to facilitate suture placement. A demarcation line 72 indicates the stitching boundary at the inflow end 60.

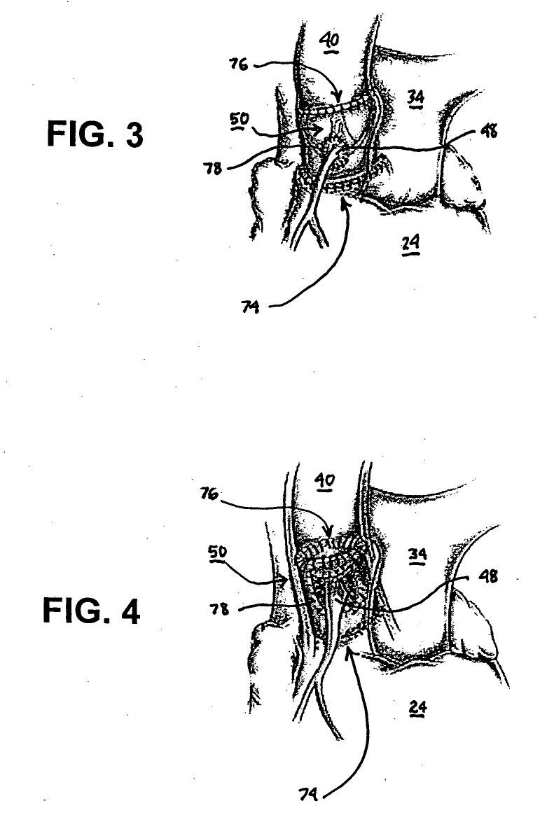

The total root, stentless tissue valve 50 allows a choice of surgical implantation techniques illustrated in FIGS. 3-6. In each instance, the valve inflow end 60 is sutured with an inflow suture band 74 of sutures to cardiac tissue surrounding the valve annulus where the native aortic valve 38 was surgically removed. A further outflow suture band 76 of sutures at the outflow end of the tissue valve 50 secures it to the ascending aorta 40. Bleeding of the high pressure arterial blood through the suture holes and along the individual sutures may be severe in certain cases as described above. In accordance with the present invention, hemostasis is achieved by obtaining a mass of fibrin sealant or platelet concentrate, applying the mass onto a porous fabric, whereby the mass is supported in the interstices or pores of the fabric, and applying the supported mass against the site of high pressure blood leakage, that is along the inflow suture band 74 and in certain cases along the outflow suture band 76. The supported mass achieves hemostasis as the fibrin sealant or platelet concentrate does not wash away from the site.

In FIG. 3, a full-root technique is depicted wherein an aorotomy is performed including isolation of the coronary arteries, and excision of the native aortic valve leaflets. The coronary ostia are mobilized on buttons of the aortic wall, and the remaining sinus of Valsalva tissue and diseased aortic wall are excised. Anastomosis sites are made in the tissue valve sidewall 52, and the tissue valve is positioned to be sutured in place. The inflow and outflow suture bands 74 and 76 are created, and an anastomosis of the coronary arteries, e.g., the depicted right coronary artery 48 is created, resulting in a further suture band 78 at each anastomosis.

In this procedure illustrated in FIG. 3, hemostasis is achieved by obtaining a mass of fibrin sealant or platelet concentrate, applying the mass onto a porous fabric, whereby the mass is supported in the interstices or pores of the fabric, and applying the supported mass against the site of high pressure blood leakage, that is along the inflow suture band 74 and along the outflow suture band 76.

In FIG. 4, a root-inclusion technique is depicted wherein an aorotomy is performed including isolation of the coronary arteries, and excision of the native aortic valve leaflets, and trimming of the inflow end of the ascending aorta 40 that the tissue valve 50 is to be attached to. Windows are created through the tissue valve sidewall 52, and the tissue valve is positioned within the ascending aorta 40. The inflow and outflow suture bands 74 and 76 are created, and the coronary arteries, e.g., the depicted right coronary artery 48, are passed through the windows. A further anastomosis suture band 78 is formed around each window.

In this procedure illustrated in FIG. 4, hemostasis is achieved by obtaining a mass of fibrin sealant or platelet concentrate, applying the mass onto a porous fabric, whereby the mass is supported in the interstices or pores of the fabric, and applying the supported mass against the site of high pressure blood leakage, that is along the inflow suture band 74.

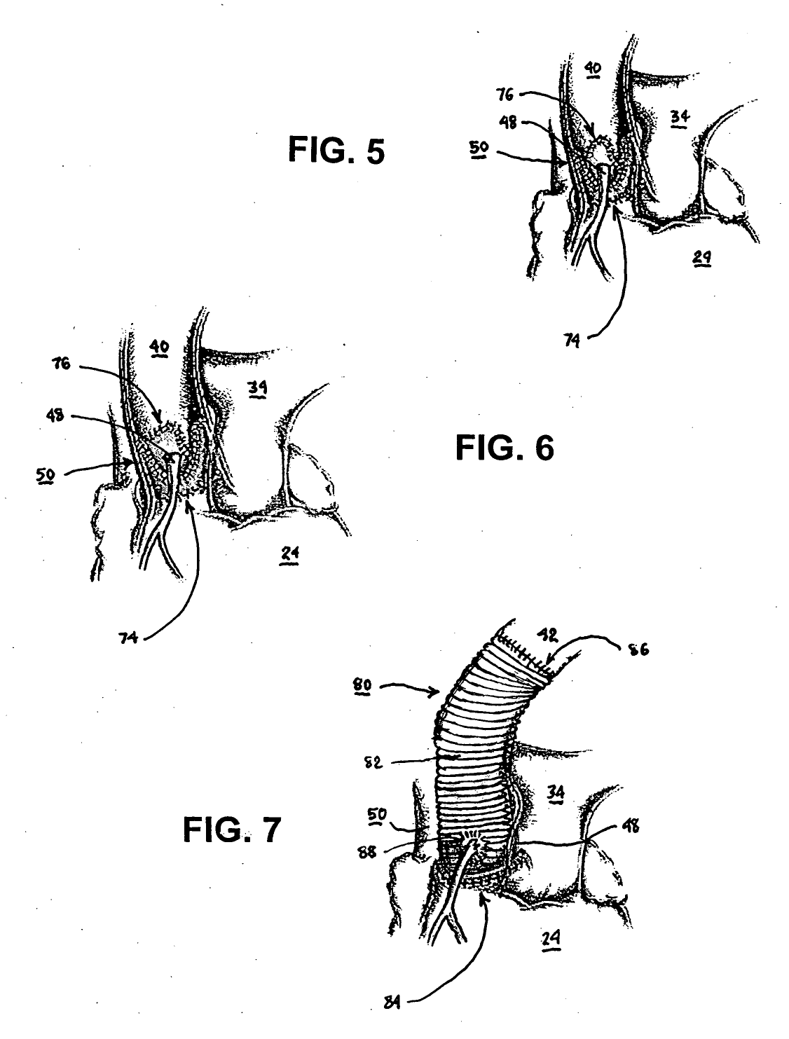

In FIG. 5, a complete subcoronary technique is depicted wherein an aorotomy is performed including excision of the native aortic valve leaflets, and trimming of the inflow end of the ascending aorta 40 that the tissue valve 50 is to be fitted within and attached to. The coronary arteries, e.g., the right coronary artery 48, remain attached to ascending aorta 40. The outflow end of the tissue valve 50 is trimmed to a scallop shape excising all three sinuses of the tissue valve 50 so as to not obstruct the ostia of the coronary arteries when inserted into the ascending aorta 40. Pre-shaped tissue valves are available that are supplied with a scalloped outflow end for fitting into the ascending aorta 40. The inflow suture band 74 and the scalloped shape outflow suture band 76 are created.

In this procedure illustrated in FIG. 5, hemostasis is achieved by obtaining a mass of fibrin sealant or platelet concentrate, applying the mass onto a porous fabric, whereby the mass is supported in the interstices or pores of the fabric, and applying the supported mass against the site of high pressure blood leakage, that is along the inflow suture band 74.

In FIG. 6, a modified subcoronary technique is depicted wherein an aorotomy is performed including excision of the native aortic valve leaflets, and trimming of the inflow end of the ascending aorta 40 that the tissue valve 50 is to be fitted within and attached to. The coronary arteries, e.g., the right coronary artery 48, remain attached to ascending aorta 40. The outflow end of the tissue valve 50 is trimmed to a scallop shape that retains the non-coronary sinus, but does not obstruct the ostia of the right and left coronary arteries when inserted into the ascending aorta 40. Pre-shaped tissue valves are available that are supplied with a scalloped outflow end for fitting into the ascending aorta 40. The inflow suture band 74 and the scalloped shape outflow suture band 76 are created.

In this procedure illustrated in FIG. 6, hemostasis is achieved by obtaining a mass of fibrin sealant or platelet concentrate, applying the mass onto a porous fabric, whereby the mass is supported in the interstices or pores of the fabric, and applying the supported mass against the site of high pressure blood leakage, that is along the inflow suture band 74.

As also noted above, various disease processes may necessitate replacement of a section of the ascending aorta 40 and/or the aortic arch 42 with an aortic graft. The surgical attachment of one end of an aortic graft 80 is depicted in FIG. 7. The aortic graft 80 is formed of a flexible fabric wall 82 extending between a graft proximal end and a graft distal end. The coronary arteries are severed from the ascending aorta 40, and the diseased section of the ascending aorta 40 superior to the leaflets of the aortic valve 38 (if not replaced by a tissue valve 50 as described above). The aortic graft proximal and distal ends are sutured to the remaining ends of the ascending aorta 40 at an inflow suture band 84 and an outflow suture band 86. An anastomosis of each end of the each coronary artery, e.g., the depicted right coronary artery 48, and the aortic graft sidewall 82 is made resulting in anastomosis suture bands, e.g., the depicted anastomosis suture band 88.

In this procedure illustrated in FIG. 7, hemostasis is achieved by obtaining a mass of fibrin sealant or platelet concentrate, applying the mass onto a porous fabric, whereby the mass is partly embedded in the interstices or pores of the fabric, and applying the supported mass against the site of high pressure blood leakage, that is along the inflow suture band 84.

Thus, suture lines through an implantable medical device are depicted in each of the FIGS. 3-7 that can constitute the site of blood leakage and where hemostasis is necessary for at least some time period after the implantable medical device is stitched in place.

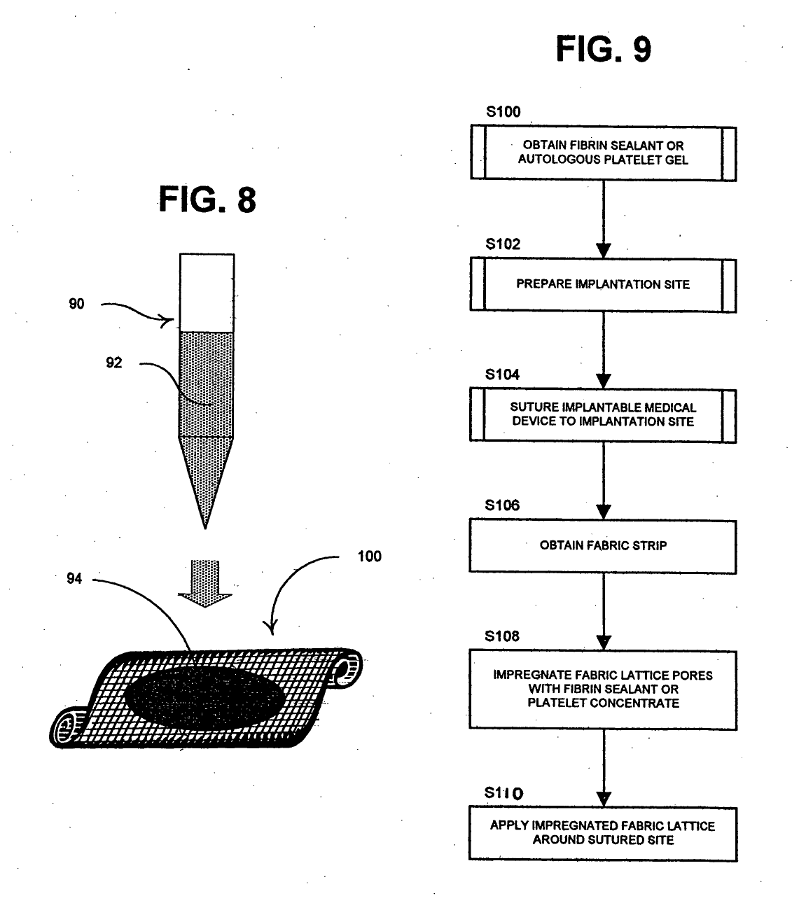

In FIG. 8, a syringe or pipette 90 filled with a fibrin sealant or platelet concentrate 92 is schematically depicted in relation to a fabric strip 100 so that a mass 94 of fibrin sealant or platelet concentrate is deposited to be supported by the fabric pores. Preferably, the fibrin sealant or platelet concentrate 92 is autogolous in nature and obtained from the patient's blood prior to or during the surgical procedure, e.g., an autologous platelet gel obtained as described above employing the Magellan™ Autologous Platelet Separator System sold by the assignee of the present invention.

The fabric strip 100 may comprise a porous material that may be flexible or packable at the site of bleeding, including one of foamed gelatin, knitted oxidized regenerated cellulose, and other coagulant entities. The fabric strip 100 is preferably formed of surgical hemostatic felt of the type described above with respect to the above-referenced '612 patent or other surgical and orthopedic felts available from U.S. Felt Mfg. Co, Sanford, Me., for example.

FIG. 9 is a flowchart illustrating the method of providing hemostasis at a surgical site, e.g., the surgical sites of FIGS. 3-7, with the surgical felt fabric strip 100 supporting the mass 94 of fibrin sealant or platelet concentrate. Thus, in step S100, the fibrin sealant or platelet concentrate is obtained prior to or during surgery, preferably from the patient's blood. The implantation site is prepared in step S102, e.g., the sites described above with respect to FIGS. 3-7. The implantable medical device is surgically attached at the implantation site in step S104.

In step S106, each fabric strip 100 to be applied is obtained by cutting strips to size or retrieving pre-cut strips from a kit supplied with the implantable medical device, in this case a tissue valve or graft. In step S108, the mass of fibrin sealant or platelet concentrate is applied as shown in FIG. 8 onto the fabric strip 100, whereby the fabric strip 100 is impregnated with the mass 94, and the mass 94 is supported in the interstices or pores of the fabric strip 100.

In step S110, the supported mass is applied against the site of high pressure blood leakage, whereby the supported mass achieves hemostasis as the fabric supports the mass from washing away from the site. In the particular applications depicted in FIGS. 3-7, a fabric strip 100 can be applied to one or more of the suture bands 74, 76, 78 or 84, 86, 88. The applying step can comprise wrapping the impregnated fabric strip 100 about the circumference of the tissue valve 50 over the suture bands 74, 76 or about the circumference of the aortic graft 80 over the suture bands 84, 86. The relatively tight spaces may be sufficient to hold the impregnated fabric strip in place. Additional fibrin sealant or platelet concentrate and/or other hemostatic agents may be applied over the fabric strip 100 to fill the space.

It should be noted that the present invention contemplates reversing the order of steps S108 and S110 when circumstances permit.

In this way, a method of providing hemostasis at a site of high pressure blood leakage through suture holes or along sutures attaching an implantable medical device to an artery is accomplished. It will be understood that the present invention may also be practiced in at other surgical sites or traumatic injury sites to stem bleeding of high or low pressure blood.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments.

In addition, it will be understood that specifically described structures, functions and operations set forth in the above-referenced patents can be practiced in conjunction with the present invention, but they are not essential to its practice.

It is to be understood, that within the scope of the appended claims, the invention may be practiced otherwise than as specifically described without actually departing from the spirit and scope of the present invention. The disclosed embodiments are presented for purposes of illustration and not limitation, and the present invention is limited only by the claims that follow.

What is claimed is:

1. A method of providing hemostasis at a site of blood leakage comprising applying a fabric and a platelet concentrate to the site of blood leakage to provide hemostasis at the site.

2. The method of claim 1, wherein the platelet concentrate comprises an autologous platelet gel.

3. The method of claim 1, further comprising applying a fibrin sealant to the site.

4. The method of claim 1, wherein the site of blood leakage comprises sutures.

5. The method of claim 4, wherein the sutures attach an implantable medical device to an implantation site.

6. The method of claim 5, wherein the implantable medical device is a prosthetic aortic valve.

7. The method of claim 4, wherein the sutures attach a prosthetic aortic graft to the site.

8. The method of claim 1, wherein the blood leakage is high pressure blood leakage.

9. The method of claim 1, wherein the blood leakage is due to trauma.

10. The method of claim 1, wherein the fabric comprises a porous material.

11. The method of claim 10, wherein the porous material is foamed gelatin, knitted oxidized regenerated cellulose or a combination thereof.

12. The method of claim 1, wherein the fabric comprises surgical hemostatic felt.

13. The method of claim 1 wherein the fabric is biocompatible.

14. The method of claim 1 wherein the fabric is bioabsorbable.

15. A method of providing hemostasis at a site of blood leakage comprising applying a fabric and a fibrin sealant to the site of blood leakage to provide hemostasis at the site.

16. The method of claim 15, further comprising applying a platelet concentrate to the site.

17. The method of claim 16, wherein the platelet concentrate comprises an autologous platelet gel.

18. A kit comprising packaging material enclosing, separately packaged, pre-formed porous fabric strips and instructions for use according to the method of claim 1.

19. The kit of claim 18, further comprising an implantable medical device.

20. The kit of claim 18, further comprising an implantable graft.
